(19) United States
(12) Patent Application Publication
(10) Pub. No.: US 2005/0031626 A1
(43) Pub. Date: Feb. 10, 2005

(54) Binding agents with differential activity

(76) Inventor: George Telford Stevenson, Southampton (GB)

Correspondence Address: FISH & RICHARDSON P.C., 1425 K STREET, N.W., 11TH FLOOR, WASHINGTON, DC, 20005-3500, US

(21) Appl. No.: 10/484,638

(22) PCT Filed: Jul. 30, 2002
(86) PCT No.: PCT/GB02/03455

(30) Foreign Application Priority Data
Jul. 31, 2001 (GB) 0118662.6

(51) Int. Cl. A61K 39/395

(52) U.S. Cl. 424/178.1; 530/387.3

(57) ABSTRACT

Binding agents with differential activity can be provided, whereby certain activities of a first part of the binding agent are reduced or prevented until binding to a target occurs. This is useful if the binding agent is intended to bind both an effector cell and a target to be destroyed, because the effector cell can be protected from significant cell damage that might otherwise occur (e.g. due to premature activation of complement and/or ADCC). Such binding agents are useful in the treatment of cancer, for example.

### CLAIM OF PRIORITY

This application claims priority under 35 U.S.C. § 371 to PCT Application Serial No. PCT/GB02/03455, filed on Jul. 30, 2002, which claims priority to Application Serial No. GB 0123260.2, filed Sep. 27, 2001, and Application Serial No. GB 0118662.6, filed on Jul. 31, 2001, each of which is incorporated by reference in its entirety.

### TECHNICAL FIELD

The present invention relates to binding agents, especially to binding agents useful in targeting cancer cells or other biological targets. In particular, the invention relates to antibodies and antibody constructs, especially bispecific antibody constructs.

### BACKGROUND

Antibodies and derivatives thereof have been used to target biological targets, including cancer cells, for many years. The predominant class of antibody is the IgG class, which has 3 globular modules, 2 Fab and one Fc, joined by an extended and flexible hinge. The Fab modules each display an integral antigen-binding site, while the Fc is responsible (a) for recruiting the molecular and cellular effectors needed to destroy an antibody-coated target cell and (b) for directing certain trafficking and metabolic characteristics of each antibody class. The cells recruited by the Fc module display molecules called Fc-receptors (FcR), which dock at sites on the surface of the Fc.

A mouse IgG1 molecule is shown in FIGS. 1A and 1B. FIG. 1A illustrates the disposition of chains and interchain disulfide (SS) bonds. Chains (2 light, 2 heavy, N termini at the top, C termini at the bottom) are represented by black ribbons. Sets of interchain noncovalent bonds are depicted by hatched rectangles, and the two antibody sites by dashed arcs. Human IgG differs only in having 2 rather than 3 inter-heavy chain SS bonds.

FIG. 1B shows a 2-dimensional diagrammatic representation of the overall protein conformation. Antibody sites are represented by triangular indentations, and noncovalent interactions between the chains by dashed lines. The chains are seen to be organized into 3 globular regions joined by a hinge comprising an extended sequence of each heavy chain. The Fc region displays sequences for recruitment of effector molecules (a set known as complement) and effector cells (chiefly macrophages and NK lymphocytes). The Fc contains a further set of sequences which prolong the metabolic life of the IgG molecule by sequestering it away from lysosomal enzymes.

Soon after monoclonal antibody technology was described in the mid-70s antibodies, generally of the IgG class, were tried in the treatment of cancer, being aimed at molecules on the surfaces of the tumour cells. For about 15 years little success was achieved. Some of the reasons for this are set out below:

(a) The docking sites for FcRI, II and III on mouse Fc have low and variable affinities for human effector cells (G T Stevenson. Immunotherapy of Tumours in Clinical Aspects of Immunology, ed P J Lachmann et al, Blackwell Scientific Publications, 1993, pp 1799-1830), which are now thought to be the principal agents involved in destroying antibody-coated tumour targets (R Clynes et al. Inhibitory Fc receptors modulate in vivo cytoxicity against tumor targets. Nature Medicine 6:443, 2000).

(b) The docking site on mouse Fc for FcRn has no detectable affinity for human FcRn, so the therapeutic mouse IgG antibody has a very short survival in man.

(c) The mouse IgG molecule is seen as foreign by the human immune system, which after about 10 days often produces antibodies against it, frequently against its Fc zone, thus further shortening the survival of the mouse antibody in the human host.

These difficulties were largely overcome by the advent of chimeric antibodies that retained mouse amino acid sequences in their antigen-binding sites, but had human IgG sequences for all or much of the remainder of the antibody. The recruitment of human effector cells and metabolic survival in the human host were thereby improved, while any immune response to the antibody was on a much less serious scale. Several chimeric antibodies are now licensed for clinical use but their effectiveness still leaves much to be desired. (D G Maloney et al. IDEC-C2B8 (rituximab) anti-CD20 monoclonal antibody therapy in patients with relapsed low-grade non-Hodgkin's lymphoma. Blood 90:2188-2195, 1997). This is because cancer cells, like mammalian cells in general, have a variety of good defences against antibody attack, these apparently having evolved to deal with untoward auto-immune responses.

Many attempts have been made to improve the efficacy of antibody attack and so to overwhelm or circumvent the defences of cancer cells. Prominent among these have been bispecific antibodies, which have two different rather than identical antibody sites. One site remains specific for a tumour while the other has been used for a variety of purposes. Frequently it targets an FcR on a human effector cell. The effector cell is thereby drawn into contact with the tumour cell and if suitably activated will destroy it. Effector cell recruitment by this means is much more tenacious than the normal recruitment by Fc. The affinity of the antibody site for the FcR (K_{A}=10⁷-10⁸) is typically about 100-fold greater than the Fc-FcR affinity.

The two most common designs for bispecific antibodies are shown in FIGS. 2A and 2B. Each has been the subject of clinical trials, but with only limited success. The construct shown in FIG. 2A binds only weakly to tumour cells and has a limited half-life of only about 24 hours. The construct shown in FIG. 2B also binds only weakly to tumour cells. Additionally, if attached to effector cells only, it can cause damage due to recruitment of effectors by the Fc portion of the construct.

From the foregoing discussions it will be appreciated that there is a great need to is develop improved binding agents against biological targets, especially binding agents useful in the treatment of cancer.

### SUMMARY

According to a first aspect of the present invention, we provide a binding agent comprising: (a) a first part that comprises one or more of the biological activities of an antibody Fc region when the binding agent is bound to a biological target; (b) a second part that is capable of binding to the biological target with a valency of two or more; and (c) a third part that is capable of monovalent binding to an effector cell so that the effector cell can act upon the biological target when the second part is bound to the biological target.

In a preferred embodiment, the effector cell is capable of destroying, damaging, altering or removing the biological target. Preferably, the biological target is deleterious to a human or non-human animal. Preferably, the biological target is a cancer cell or a part thereof.

Preferably, at least one of the biological activities of the first part is modulated when the binding agent is bound to the biological target in comparison with when the binding agent is bound to the effector cell only.

More preferably, at least one of the biological activities of the first part is at least ten times higher when the binding agent is bound to the biological target in comparison with when the binding agent is bound to the effector cell only.

The binding agent may be such that, in the absence of binding of the second part to the biological target, the binding agent is configured so that at least one biological activity of the first part is prevented or reduced due to steric hindrance, and in which the steric hindrance is removed or reduced when the second part binds to the biological target.

Preferably, the first part comprises an FcRn docking site that is not sterically hindered in the absence of binding of the second part to the biological target. Preferably, the first part comprises one or more of the following biological activities when the binding agent is bound to a biological target: (a) complement activation; and (b) binding to the neonatal or Brambell Fc-receptor (FcRn).

In a preferred embodiment, the binding agent is modified to reduce activation of an effector cell in the absence of binding of the binding agent to a biological target. Preferably, it is modified to reduce binding to an FcRI, FcRII and/or an FcRIII receptor in the absence of binding of the binding agent to a biological target.

Preferably, the first part comprises an Fc region which lacks one or more glycans, preferably two glycans, normally associated with a natural Fc molecule. Preferably, the glycans are those linked to asparagine corresponding to position 297 of the IgG heavy chain. Preferably, the first part comprises an Fc region which is enzymatically deglycosylated, preferably with glycoamidase PNGaseF.

Alternatively, or in addition, the first part comprises a recombinant Fc region in which the asparagine residue corresponding to position 297 of the IgG heavy chain is replaced with a non-gylcosylatable amino acid residue.

In another embodiment, the first part further comprises one or more of the following biological activities when the binding agent is bound to a biological target: (c) induction or stimulation of phagocytosis by phagocytic cells; and (d) antibody-dependent cellular cytotoxicity (ADCC). Futhermore, the at least one biological activity may include binding with FcRI, FcRII and/or FcRIII receptors.

Preferably, endosomal binding to the first part so as to reduce lysosomal degradation of the binding agent in vivo is not prevented.

Preferably, the second part is capable of binding to a plurality of different biological targets or to a plurality of different parts of the same biological target.

In preferred embodiments, the binding agent comprises one or more Fab, Fab′ or F(ab′)₂ regions or parts thereof. Preferably, it comprises one or more Fc regions, or parts thereof. Preferably, it comprises at least one anti-target Fab, Fab′ or F(ab′) ₂ regions or parts thereof, at least one anti-effector cell Fab, or Fab′ regions or parts thereof, and at least one Fc region or a part thereof. In preferred embodiments, the binding agent comprises at least two anti-target Fab, Fab′ or F(ab′)₂ regions or parts thereof

Preferably, any one or more of the first, second and third parts of the binding agent are derived from an IgG molecule. Any one or more of the first, second and third parts may be covalently linked to each other. The binding agent may comprise one or more tandem thioether links that interconnect cysteine residues.

Preferably the second part binds specifically to the biological target. Preferably, the second part comprises anti-CD 20 and/or anti CD-37 binding activity.

Preferably, the third part binds specifically to the effector cell. Preferably, the third part comprises anti-CD 16 binding activity.

The binding agent may comprise a modular structure, in which one module is capable of binding to a biological target, one module is capable of binding to an effector cell and another module comprises one or more of the biological activities of an antibody Fc region when the binding agent is bound to a biological target. Preferably, it comprises two modules capable of binding to the same biological target.

We provide, according to a third aspect of the present invention, a binding agent as described, when bound to an effector cell.

As a fourth aspect of the present invention, there is provided a part, component or module of a binding agent, for use in the manufacture of a binding agent as described.

We provide, according to a fifth aspect of the present invention, a method of providing a binding agent, comprising providing a plurality of modules and connecting them via tandem thioether linkages between cysteine residues.

There is provided, according to a sixth aspect of the present invention, a method of providing a binding agent, comprising the steps of: (a) providing a first part comprising one or more of the biological activities of an antibody Fc region when the binding agent is bound to a biological target; (b) providing a second part capable of binding to the biological target with a valency of two or more; (c) providing a third part capable of monovalent binding to an effector cell so that the effector cell can act upon the biological target when the second part is bound to the biological target; and covalently joining the first, second and third parts.

Preferably, the modules or parts of the binding agent are as set out in any preceding aspect of the invention. Preferably, the modules or parts are linked via a maleimide linker (e.g. o-phenylenedimaleimide (PDM)).

In a seventh aspect of the present invention, there is provided a binding agent according to any preceding aspect of the invention for use in medicine.

According to an eighth aspect of the present invention, we provide the use of a binding agent in the preparation of a medicament for treating a disease or disorder caused by or involving the biological target. Preferably, the disease or disorder is selected from the group consisting of: cancer, including a lymphoma (e.g. a B-cell lymphoma), an infectious disease or disorder and an autoimmune disease or disorder.

There is provided, in accordance with a tenth aspect of the present invention, a pharmaceutical composition comprising a binding agent as described; the composition optionally comprising a pharmaceutically acceptable carrier, diluent or excipient.

As an eleventh aspect of the invention, we provide an image or model, preferably a computer generated image or model, of a binding agent as described. We provide, according to a twelfth aspect of the invention, there is provided a data carrier that comprises data for such an image or model. According to a thirteenth aspect of the present invention, we provide a computer that comprises data for such an image or model, and/or that comprises such a data carrier.

We provide, according to a fourteenth aspect of the invention, there is provided a method comprising providing an image or model as described, a data carrier as described, or a computer as described and using it to predict the structure and/or function of potential new therapeutic binding agents. Preferably, the method comprises making one or more changes to the image or model and, optionally, predicting or analysing an effect of those changes.

According to a fifteenth aspect of the present invention, we provide a drug development program that uses a binding agent as described, an image or model as described, a data carrier as described, a computer as described, or a method as described.

According to a sixteenth aspect of the present invention, we provide a drug or drug candidate obtained or identified using such a drug development program.

There is provided, according to a seventeenth aspect of the present invention, a method comprising providing a binding agent as described, or a drug or drug candidate as described and testing in vivo or in vitro the activity and/or binding of the binding agent, drug, or drug candidate against a biological target.

We provide, according to a eighteenth aspect of the present invention, a method comprising providing a binding agent as described, or a drug or drug candidate as described and testing in vivo or in vitro the toxicity of the binding agent, drug, or drug candidate.

According to a nineteenth aspect of the present invention, we provide a binding agent as described, or a drug or drug candidate as described, when in immobilised form.

According to a twentieth aspect of the present invention, we provide an array comprising a binding agent as described, or a drug or drug candidate as described.

There is provided, according to a twenty-first aspect of the present invention, a method comprising the steps of: (a) exposing a Fc-containing polypeptide to a matrix; (b) allowing the Fc-containing polypeptide to bind to the matrix by a hydrophobic interaction; (c) removing the Fc-containing polypeptide from the matrix by disrupting the hydrophobic interaction.

We provide, according to a twenty-second aspect of the invention, a method of separating an Fc-containing polypeptide from other components in a sample, the method comprising: (a) exposing the sample to a matrix; (b) allowing the Fc-containing polypeptide to bind to the matrix by a hydrophobic interaction; and optionally removing one or more components of the sample by washing the matrix; and (c) removing the Fc-containing polypeptide from the matrix by disrupting the hydrophobic interaction.

Preferably, the matrix comprises Toyopearl TSK-butyl-650.

In many cases the target will be deleterious to a human or non-human animal. It is therefore preferred that the effector cell is capable of destroying, damaging, altering or removing the target. The effector cell may do this directly and/or indirectly (e.g. via the recruitment of other moieties, such as cytokines or other cells).

As used herein, an “effector cell” may thus be any cell capable of giving rise to or promoting a desired biological effect. Preferred cells include cytotoxic T-cells, natural killer (NK) cells, monocytes and dendritic cells.

The effector cell is advantageously protected from the antibody Fc biological activities mediated by the first part of the molecule. In a preferred embodiment, the activity of the Fc module is hindered in the absence of target binding, for example by steric means, such that the antibody Fc activity of the binding agent-effector cell complex is relatively low in the absence of target cell binding. Advantageously, at least one antibody Fc activity is increased by 10 times on target binding, preferably by 20 times, 40 times, 60 times, 100 times or more. In a further preferred aspect more than one antibody Fc activity may be so increased, for example 2, 3, 4 or more such activities.

In a preferred aspect, the binding agent is constructed by covalently linking together antibody fragments. Preferably, the first part comprises an antibody Fc region, and the second and third parts are selected from antibody binding fragments, such as Fv, scFv, Fab, F(ab′)₂ and Fab′. The fragments are linked together advantageously via a maleimide linker, such as o-phenylenedimaleimide.

The methods and compositions described here are variously applicable to medicine, as described below, including veterinary medicine and diagnostics.

The molecules as described here may moreover be represented in silico for use in molecular modelling and drug design. Thus, we provide in silico models of the molecules as described. In the above aspects, preferably a structural model of the binding agent is generated using molecular modelling techniques. Advantageously these are computer implemented modelling techniques. Suitable programs include grid-based techniques and/or multiple copy simultaneous search (MCSS) methods. These will be familiar to those skilled in the art. Alternatively, visual inspection of a computer model of a binding agent can be used, in association with manual docking of models of functional groups into its binding pockets.

Once a structural model has been generated as herein described, possible targets and/or effector groups to simulate or modulate target binding may for example be identified by one or more of the following techniques: de novo compound design, by defining a pharmacophore as herein defined, and/or by using automated docking algorithms as herein described.

In the first aspect (de novo compound design) may be performed using suitable computer software. In a preferred embodiment of this aspect, the software is selected from the group consisting of: QUANTA, SYBYL, HOOK and CAVEAT. Alternatively, linking the functional groups may be performed manually.

Suitable in silico libraries for use in the methods and compositions described here will be familiar to those skilled in the art, and includes the Available Chemical Directory (MDL Inc), the Derwent World Drug Index (WDI), BioByteMasterFile, the National Cancer Institute database (NCI), and the Maybridge catalogue.

In a further aspect, we provide a compound identifiable using one or more of the methods as described here.

In a further aspect still, we provide a computer readable medium for a computer, characterised in that the medium contains the atomic-co-ordinates of a binding agent as described herein.

The methods and compositions described here may employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

The details of one or more embodiments are set forth in the accompanying drawings and the description below. Other features, objects, and advantages will be apparent from the description and drawings, and from the claims.

### DESCRIPTION OF DRAWINGS

FIGS. 1A and 1B show schematic representations of a mouse IgG1 molecule. FIG. 1A illustrates the disposition of chains and interchain disulfide (SS) bonds. FIG. 1B shows a 2-dimensional diagrammatic representation of overall protein conformation.

FIG. 2 shows two types of bispecific antibody construct. FIG. 2A shows a bis-Fab bispecific antibody in which the right-hand Fab targets an abnormal cell such as a tumor cell and the left-hand Fab, with a different antibody site, recruits an effector cell such as a macrophage. FIG. 2B shows an IgG bispecific antibody, usually prepared by hybridoma technology.

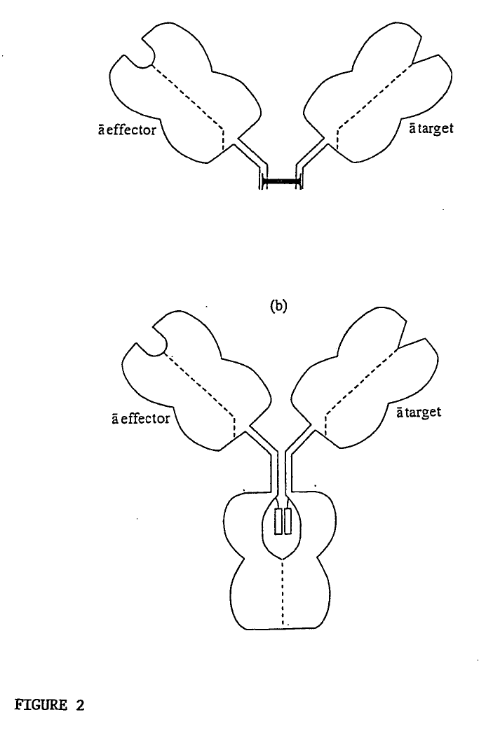

FIG. 3 shows a schematic representation of an embodiment of a differentially activated bispecific antibody.

FIG. 4 illustrates methods of joining polypeptide chains using linkers. The top two drawings show modules for engineering, with hinge-region cysteine residues displaying SH groups after reduction of interchain S—S bonds. Top left: Fab′γ from monoclonal or recombinant source. Top right: Fcγ from human normal IgG1. Lower two drawings show methods of joining two modules with the SH-directed linker o-phenylenedimaleimide (o-PDM).

Like reference symbols in the various drawings indicate like elements.

### DETAILED DESCRIPTION

A binding agent as described here comprises three parts. A first part of the binding agent has one or more of the biological activities of an antibody Fc region, when the binding agent is bound to a biological target.

The first part of the binding agent desirably has one or more of the following biological activities when the binding agent is bound to a biological target: (a) complement activation (which is achieved in mammals by binding to C1q); and (b) induction or stimulation of phagocytosis.

In certain embodiments, described in further detail later, the first part of the binding agent further comprises any one or more of the following biological activities when the binding agent is bound to a biological target: (c) antibody-dependent cellular cytotoxicity (ADCC); (d) binding to the neonatal or Brambell Fc receptor (FcRn).

Differential Activity

When the binding agent is not bound to the target, it is not essential that any or all of the biological activities of the first part are present. However, it is preferred that the ability to bind to the neonatal receptor FcRn be present in a binding agent when it is not bound to the target.

Indeed it is preferred that one or more of these activities are not present, or are present at only a relatively low level. A relatively low level may, for example, be considered to be less than 50%, less than 10%, or less than 1% of the level of activity present when the binding agent is bound to the target. In natural antibody molecules (IgG) the activation of biological activities is dependent on aggregate binding to form arrays of Fc portions; binding to effector cells may also up regulate such activities. In one embodiment of the construct as described here, activities (a) to (c) above are hindered in the event that only the effector cell is bound, and are dependent on target binding.

In a preferred embodiment, the cell lysis activity of the binding agent when it is not bound to the target is relatively lower than when it is bound to the target. Preferably, the cell lysis activity of the first part is at least ten times higher when the agent is bound to the target in comparison with when the agent is bound to the effector cell only. Preferably, the cell lysis activity is at least 10 times higher. Preferably, the titre of the binding agent which induces 50% of plateau cell lysis is at least 5 times less, preferably 10 times less, more preferably 50 times or 100 times less, when it is bound to the target compared to when it is bound to the effector cell only.

Thus, the methods and compositions described here allows for “differential activity”, whereby certain activities of the first part of the binding agent are reduced or prevented until binding of the second part to the target occurs.

The property of differential activity is useful when the effector cell is bound to the binding agent but the target is not bound, because the effector cell can be protected from significant Fc mediated cell damage that might otherwise occur (e.g. due to premature activation of complement and/or ADCC). However relatively high activity against a target can still be provided when the target is bound to the binding agent.

Previously, there was no consideration in the art of providing binding agents with such differential activity. Indeed prior art binding agents that were designed to bind both effector cells and cancer cell targets often suffered from the disadvantage that complement activation and/or ADCC occurred at an early stage so that effector cells were destroyed or damaged before being brought into the proximity of target cells. The provision of the binding agents as described here therefore represents a major advance in the field of immunotherapy.

Without being bound by theory, it is believed that differential activity is achieved due to steric hindrance of one or more sites present on the first part of the binding agent when the binding agent is bound to an effector cell but not to the target; Removal of or reduction in steric hindrance is thought to occur when the second part of the binding agent binds to the target. More specifically, it is believed that the second part of the binding agent may, in its unbound state, cause steric hindrance of one or more functional sites present on the first part of the binding agent and that a conformational change occurs on binding to the target so as to remove or reduce steric hindrance of said one or more sites of the first part. Furthermore, in embodiments where the second part has a valency of two or more, the differential valency in the molecule for target and effector cell epitopes may also contribute to the differential activity.

First Part of Binding Agent

In a highly preferred embodiment, the first part comprises at least a portion of a Fc region of an immunoglobulin, preferably, an Fc region of an IgG antibody.

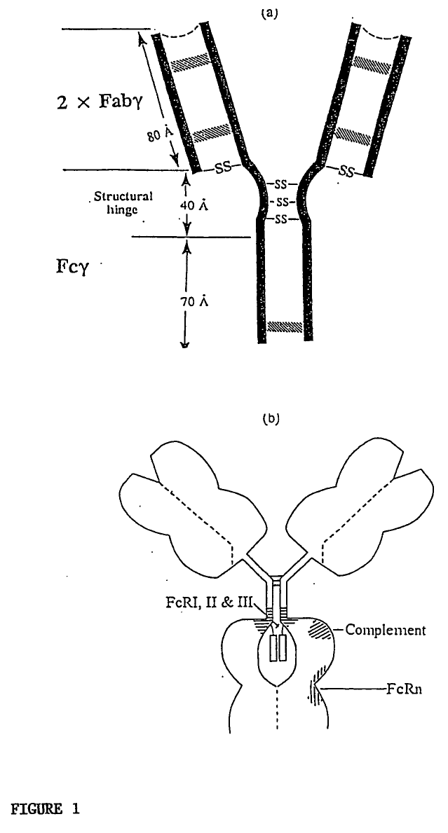

The Fc region displays sequences for recruitment of effector molecules (a set known as complement) and effector cells (chiefly macrophages and NK lymphocytes). The Fc contains a further set of sequences which prolong the metabolic life of the IgG molecule by sequestering it away from lysosomal enzymes. The space between the 2 heavy chains at the hinge-proximal region is filled by carbohydrate in the form of 2 oligosaccharide chains, depicted in FIG. 1 by rectangles, attached to asparagine 297 on each heavy chain.

Docking sites for Fc interactions are indicated by hatched areas in FIG. 1. (A mirror-image second set of sites, reflecting the symmetry of the molecule, is not shown.) Effector cells such as macrophages display 3 classes of receptor for the Fc of IgG: FcRI, II and III. These receptors compete for a common site involving and adjacent to the lower hinge, a cup-like area kept patent by the carbohydrate chains. The site for complement is also seen to be near the lower hinge in FIG. 1. The receptor for prolonging metabolic survival, the neonatal Fc-receptor (FcRn) or Brambell receptor, found on a variety of cells throughout the body, has a docking site further towards the C-terminus of the heavy chain.

The docking site for FcRI, II and III has been visualized by X-ray crystallography (P Sondermann et al. The 3.2-Å crystal structure of the human IgG1 Fc fragment-FcγRIII complex. Nature 406:267-273, 2000), as has the site for FcRn (W L Martin et al. Crystal structure at 2.8Å of an FcRn/heterodimeric Fc complex: mechanism of pH-dependent binding. Molecular Cell 7:876-77, 2001). The complement site has been deduced from site-directed mutagenesis, with the details subject to some ambiguity (A R Duncan & G Winter. The binding site for C1q on IgG. Nature 332:738-740, 1988; M H Tao et al. Structural features of human immunoglobulin-G that determine isotype-specific differences in complement activation. J. Exp. Med. 178:661-667, 1993).

FcRn Site

The FcRn docking site is distinct from the shared site for the FcRI, FcRII and FcRIII families on effector cells (FIG. 1). FcRn is present on a variety of cells and is called the “neonatal Fc-receptor”, because it is involved in the transport of maternal IgG across the placenta and across the neonatal gut. In addition to these transport functions FcRn is responsible for the prolonged metabolic survival of the IgG molecule. In man IgG is seen to survive in the body with a half-life of about 20 days, compared with 3-4 days for the other antibody classes—a point of great therapeutic importance. FcRn is displayed on endosomes of the cells which internalize and break down plasma proteins. Most of the IgG taken up is bound to this FcRn, which returns the IgG to the bloodstream instead of letting it progress with other proteins to destruction in the cell's lysosomes.

One site that is believed to be sterically hindered when the binding agent binds to an effector cell (prior to binding to the target) is present on the Fc region of IgG and is involved in the induction of complement mediated cell damage when the binding agent is bound to the target. In humans this site is the C1q binding site.

Another site that is believed to be sterically hindered is the site that binds to FcRI, FcRII and FcRIII receptors.

In contrast, the FcRn binding site (involved in binding to an endosomal receptor so as to prevent or reduce lysosomal degradation) is believed not to be substantially sterically hindered. The absence of substantial steric hindrance at this site is believed to be of great advantage in increasing the half-life of the binding agents when in use. Preferably the half-life in humans is at least 1 day. More preferably, it is at least 2, at least 4, or at least 8 days. Most preferably, it is at least 16 days.

Preferred binding agents comprise a target-binding part with a higher binding valency than the effector cell-binding part. For example, the target-binding part may bind to two, three or four epitopes (which may be the same or different) and the effector cell-binding part may bind to fewer epitopes (e.g. to a single epitope). In a preferred embodiment, the target-binding part binds to two epitopes and the effector cell-binding part binds to one epitope.

Although preferred embodiments comprise target-binding parts with a valency of two or more, univalent binding agents are also envisaged. Such a construct may comprise one instead of two Fab(anti-target) modules, and may be useful in certain situations. There is a minority of cell-surface molecules which respond to cross-linking by undergoing extremely rapid modulation, a process of redistribution and internalization which assists the cell in resisting attack by any antibody aimed at that set of molecules. With such a target, univalent antibody has been shown to be more effective than bivalent antibodies (M J Glennie & G T Stevenson. Univalent antibodies kill tumour cells in vitro and in vivo. Nature 295:712-714, 1982).

Desirably binding is specific so that the effector and target-binding parts do not cross-react with undesired moieties when binding the effector and target respectively. For example, the effector-binding part may have specific anti-CD16 binding activity and the target-binding part may have specific anti-CD20 and/or anti-CD37 binding activity.

In some cases the binding agent may be provided in a form in which it is already bound to an effector cell. Alternatively it may be provided in unbound form and may bind an effector cell in vivo.

In a preferred embodiment, the first part of the binding agent is modified such that its binding to Fc receptors is reduced or impaired. Preferably, the first part does not comprise Fc receptor binding activity. Preferably, the Fc receptor is selected from one or more of FcRI, FcRII and FcRIII receptor families.

Reduction or abolition of Fc binding activity is preferably accomplished by removal of one or more oligosaccharides or glycans normally associated with a natural IgG Fc region. Preferably, a glycan covalently linked to the Fc region is removed. Preferably, the glycan comprises an asparagine linked glycan, more preferably, a glycan attached to an asparagine residue corresponding to residue number 297 on the IgG heavy chain.

Deglycosylation may be accomplished enzymatically, e.g., with a suitable oligosaccharide cleaving enzyme such as an amidase or glycoamidase. Although any suitable glycoamidase may be used, a preferred enzyme is peptide-N⁴-(N-acetyl-β-glucosaminyl)asparagine amidasePNGase F (EC 3.5.1.52), described in detail in Tarentino and Plummer (1994, Meth Enz 230, 44-57). The binding agent may be reacted with the enzyme before during or after its component parts are put together, i.e., the glycan may be removed from the Fc region before or after linkage to any of the Fab components. Preferably, the Fc component comprising the first part of the binding agent is treated with enzyme prior to being assembled.

Furthermore, it will be appreciated that glycan removal may be effected by genetic means. Thus, a mutation may be introduced into a coding sequence for the Fc region, or a heavy chain encoding sequence. A suitable mutation replaces the asparagine residue in the encoded amino acid with a non-glycosylatable residue, such as a residue other than serine or threonine, preferably an alanine residue.

Immunoglobulins

Preferred binding agents are derived from IgG molecules or parts thereof, although other immunoglobulins or parts thereof may be used, if desired.

IgG molecules (or other Ig molecules) may be obtained as monoclonal or polyclonal antibodies. For example, polyclonal antibodies can be obtained by purifying them from a human or animal host after the host has been exposed to an immunogen. If desired, an adjuvant may also be administered to the host to aid in immunostimulation. Well-known adjuvants include Freund's adjuvant (complete or incomplete) and aluminium hydroxide. Monoclonal antibodies can be produced from hybridomas. These can be formed by fusing together myeloma cells and spleen cells that produce a desired antibody in order to form an immortal cell line. Thus the well-known Kohler & Milstein technique (Nature 256 (1975)), or subsequent variations upon this technique can be used.

More recently, techniques such as ‘phage display have been used to express antibodies. These techniques are becoming increasingly popular and are described for example by M J Geisow in Tibtech 10, 75-76 and by D. Chiswell et al in Tibtech 10, 8-84, (1992). They can be used to express antibodies recognising desired epitopes.

Antibodies can be purified by various techniques, including adsorption to staphylococcal protein A. The staphylococcal protein will usually be coupled to a solid support, such as Sepharose beads. This can be done via cyanogen bromide coupling. Antibodies bind to protein A chiefly by hydrophobic interactions, which can be disrupted when desired so as to elute the antibodies (e.g. via transient exposure to low pH). Antibodies binding to known epitopes can be purified by elution using the epitopes in immobilised form to select the antibodies, followed by elution with an appropriate buffer.

If desired, antibodies can be provided in forms not occurring in nature—i.e. in synthetic form. Thus, for example, humanised (or primatised) antibodies may be provided. An example of a humanised antibody is an antibody having human framework regions, but rodent hypervariable regions. Methods for producing chimeric antibodies are discussed for example by Morrison et al in PNAS, 81, 6851-6855 (1984), by Takeda etalin Nature. 314, 452-454 (1985) and by Cunningham et al in Tibtech 10, 112-113 (1992). Synthetic antibody constructs are also discussed by Dougall et al in Tibtech 12,372-379 (September 1994).

In summary, techniques for producing and purifying antibodies of various kinds are well known in the art. They are discussed in standard immunology textbooks—e.g. in Roitt, I. M. et al. (1998, Immunology, 5^{th} Edition, Mosby International Ltd).

Binding agents can be obtained by providing parts of antibodies and linking them together covalently. The parts may be obtained via enzymatic cleavage (although other techniques including chemical synthesis and genetic engineering may be used). For example, papain cleavage can be used to provide two Fab fragments and an Fc fragment from an IgG molecule. Alternatively, pepsin cleavage can be used to provide an F(ab′)₂ fragment and a pFc′ fragment from such a molecule. Chemical reduction of the F(ab′)₂ fragment can then be used to provide two Fab′ fragments. Roitt et al (supra) describes immunoglobulin structure and function in detail, including the provision of the aforesaid fragments.

A binding agent of preferably comprises a plurality of Fab, Fab′ or F(ab′)₂ regions or parts thereof. Desirably it also comprises one or more Fc regions, or parts thereof.

For example, the binding agent may comprise at least two anti-target antigen binding regions or parts thereof (e.g. at least two anti-target Fabγ′ fragments); at least one anti-effector cell antigen binding region or part thereof (e.g. at least one anti-effector Fabγ′ fragment); and at least one Fcγ fragment or part thereof. If desired, the anti anti-target binding regions (or at least the CDR segments thereof) may be derived from a different species from the species for which the binding agent is to be used in treatment. It is however preferred that part or all of the Fc regions is derived from the same species as that for which the binding agent is to be used in treatment. Thus the binding agent may be chimeric and may be humanised.

It is of course possible to use variants of the specific fragments described above, whilst still retaining desired functional properties. Such variants are within the scope of the methods and compositions described here.

For example, a skilled person is aware that various amino acids have similar characteristics and that one or more amino acids can often be substituted by one or more other such amino acids without eliminating a desired property of a polypeptide. Substitutions of this nature are often referred to as “conservative” or “semi-conservative” substitutions. For example, the amino acids glycine, alanine, valine, leucine and/or isoleucine can often be substituted for one another (amino acids having aliphatic side chains). Of these possible substitutions it is preferred that glycine and alanine are used to substitute for one another (because they have relatively short side chains) and that valine, leucine and isoleucine are used to substitute for one another (because they have larger aliphatic side chains which are hydrophobic). Other amino acids that can often be substituted for one another include phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and/or cysteine and methionine (amino acids having sulphur-containing side chains).

Another possibility is to provide one or more deletions. This can be advantageous because the overall length and the molecular weight of a polypeptide can be reduced, whilst still retaining a desired property. Thus, if desired, non-essential or non-important sequences may be removed from a given polypeptide.

Amino acid insertions can also be made. This may be done to alter the nature of the polypeptide (e.g. to assist in identification, purification or expression.).

Whatever amino acid changes may be made, it is preferred to retain at least 40% sequence identity for the appropriate part of a binding agent as described with the sequence of a naturally occurring Fab, Fab′, F(ab′)₂, Fc, or Fc′ region. More preferably, the degree of sequence identity for said part with said region is at least 50%, at least 60%, at least 70%, or at least 80%. High sequence identities of at least 90%, at least 95%, or at least 99% are most preferred. The percentage sequence identity between two amino acid sequences can be determined in a number of ways. For example:

1) It can be determined in a simple manner by aligning a given sequence with a reference sequence without allowing for gaps in a manner so that the maximum number of amino acids or nucleotides match up with each other. The percentage sequence identity (S) can then be calculated by using the equation: S=100×(M/N); where M is the number of nucleotides or amino acids in the given sequence that are identical with nucleotides or amino acids at the corresponding positions in the reference sequence and N is the total number of amino acids or nucleotides in the reference sequence.

If gaps are allowed then gap penalties may be incurred. For example, gaps may be penalised in a simple manner simply by considering the gaps to represent amino acid or nucleotide mismatches over the full length of any gaps present. Two gaps of 5 and 10 amino acids would therefore be considered to represent mismatches totalling 15 amino acids and the proportion of matches would be reduced (relative to a system in which gaps were not penalised). More sophisticated systems for penalising gaps are also known and may involve separate penalties for gap lengths and for the numbers of gaps present.

2) It can be determined by using various algorithms, which may be incorporated in computer programs. (If desired, the default parameters of these programs can be used.).

For example, the algorithm of Karlin and Altschul (1990 Proc. Natl. Acad. Sci. USA 87:2264-68), or a modified version thereof can be used (see e.g. Karlin and Altschul 1993 Proc. Natl. Acad. Sci. USA 90:5873-77). This algorithm is incorporated into the NBLAST and XBLAST programs (version 2.0) of Altschul, et al. (1990 J. Mol. Biol. 215:403-10.) Nucleotide searches can be performed with the NBLAST program (e.g. score=100, wordlength=11). BLAST protein searches can be performed with the XBLAST program (e.g. score=50, word length=3). To obtain gapped alignments for comparison purposes, Gapped BLAST can be utilised as described in Altschul et al., 1997 Nucleic Acids Research 25(17):3389-3402. When utilising BLAST and gapped BLAST programs it is preferred that the default parameters of the respective programs (e.g., XBLAST and NBLAST) are used (see e.g. http://www.ncbi.nlm.nih.gov/BLAST).

Alternatively, the FASTA program can also be used. This is based upon a modified version of the Wilbur and Lipman algorithm (see e.g. http://www2.ebi.ac.uk/fasta 3).

Another algorithm that can be used is that of Myers and Miller, (CABIOS, 4:11-17 (1989)).

This algorithm is incorporated into the ALIGN program (version 2.0) which is part of the GCG sequence alignment software package (see e.g. http://www2.igh.cnrs.fr/bin/align-guess.cgi). When using the ALIGN program for comparing sequences, a PAM 120 weight residue table, a gap length penalty of 12, and a gap penalty of 4 can be used, for example.

For the purposes of the methods and compositions described here, it is preferred that sequence identity is determined by using Gapped BLAST (version 2.0), using the default parameters provided.

Different parts of a binding agent as described may be linked together covalently. Desirably one or more tandem thioether links are used to link cysteine residues, so as to link covalently different parts at or proximal to a hinge structure. Details of the provision of thioether links and hinge structures are provided by Stevenson (Antibody Engineering, Chem. Immunol., Basel, Karger, 1997, 65, 57-72).

The linking together of parts is preferably used to provide a binding agent having a tetramodular structure, whereby two modules are capable of binding to a biological target (or to two different biological targets), one module is capable of binding to an effector cell and another module has one or more of the biological activities of an antibody Fc region when the binding agent is bound to the biological target.

Binding agents may be provided in substantially pure form if desired. The term “substantially pure form” is used to indicate that a given component is present at a high level. The component is desirably the predominant protein component present in a composition. Preferably it is present at a level of more than 75%, of more than 90%, or even of more than 95%, said level being determined on a dry weight/dry weight basis with respect to the total protein composition under consideration. At very high levels (e.g. at levels of more than 90%, of more than 95% or of more than 99%) the component may be regarded as being in “isolated form”. Where the proteins are present in a buffer solution, inorganic buffer salt components may be present, and if taken into account their mass may exceed that of the protein fraction and thus substantially reduce the percentage figures set forth above.

Purification of Fc Containing Polypeptides

We also provide for a novel method of purification of a polypeptide comprising a Fc region (or any substantial part of an Fc region), by separation based on protein surface hydrophobicity. Preferably, such purification is carried out by the use of hydrophobic interaction chromatography.

Thus, we provide for a method comprising the steps of: (a) exposing a Fc-containing polypeptide to a matrix; (b) allowing the Fc-containing polypeptide to bind to the matrix by a hydrophobic interaction; (c) removing the Fc-containing polypeptide from the matrix by disrupting the hydrophobic interaction. We further provide for a method of separating an Fc-containing polypeptide from other components in a sample, the method comprising: (a) exposing the sample to a matrix; (b) allowing the Fc-containing polypeptide to bind to the matrix by a hydrophobic interaction; and optionally removing one or more components of the sample by washing the matrix; and (c) removing the Fc-containing polypeptide from the matrix by disrupting the hydrophobic interaction.

Preferably, the matrix comprises one or more hydrophobic groups, for example, phenyl or butyl groups. More preferably, the matrix comprises polyhydroxymethacrylate gel bonded with phenyl or butyl group. Preferably, the matrix is in the form of a column, Preferably, step (b) is carried out in the presence of a higher salt concentration than in step (c). More preferably, step (c) is carried out by elution of the matrix with dilute aqueous salt solution, preferably in the presence of an organic solvent. A suitable organic solvent comprises 10% dimethylformamide.

According to this method, a composition containing a Fc-containing polypeptide is exposed to a suitable hydrophobic column under high salt conditions. The bound protein is then eluted with a suitable solvent (such as a decreasing salt gradient). It will be appreciated that such purification may be used as a means of increasing the concentration of the protein within a sample.

The novel technique is suitable for separating any polypeptide comprising a Fc-region from other materials, such as contaminants. It is particularly suitable for separating Fc regions for use in making the binding agents described here. A particular example of such a purification is presented in (d) of Example 5.

Suitable columns for hydrophobic interaction chromatography are known in the art, and are available for example from Amersham Biosciences (e.g., HiTrap HIC Selection Kit) and Agilent Technologies (e.g., TSK Phenyl-5PW, TSK Ether-5PW or SynChropak H-Propyl). In a preferred embodiment, the matrix comprises Toyopearl TSK-butyl-650.

Hydrophobic interaction chromatography (also known as HIC) is a technique for the purification and separation of biomolecules based on differences in their surface hydrophobicity. HIC techniques have been used as a part of protein purification strategies in combination with other chromatographic techniques such as GF and IEX, as well as an analytical tool for the detection of protein conformational changes. Many biomolecules, generally considered to be hydrophilic, also have sufficient numbers of hydrophobic groups allowing interaction with hydrophobic ligands coupled to the chromatographic matrix. While HIC and RPC are closely related techniques, adsorbents for RPC are more highly substituted with hydrophobic ligands than HIC adsorbents. This feature allows the use of mild elution conditions to help maintain the biological activity of the sample.

It will be appreciated that other purification techniques may be used to complement hydrophobic interaction chromatography of Fc containing polypeptides. Such other techniques include chromatography methods known in the art, such as ion exchange, size exclusion and affinity chromatography.

Uses

Binding agents as described in this document are particularly useful in medicine.

They can be used in the preparation of a medicament for treating a disease or disorder caused by or involving a biological target.

Treatments may benefit a human or non-human animal. Thus human and veterinary treatments are within the scope of the methods and compositions described here. The treatment of mammals is particularly preferred.

A treatment may be in respect of an existing condition or it may be prophylactic. It may be of an adult, a juvenile, an infant, a foetus, a cell, tissue, or organ, or a part of any of the aforesaid.

Binding agents as described are useful in treating cancer (e.g. lymphomas). However they can be used to treat other diseases or disorders where the target is deleterious to a human or non-human animal. Examples of such other diseases or disorders include pathogenic or autoimmune diseases or disorders, and infectious diseases wherein infected cells may be detected—such as viral infections.

Pharmaceutical Composition

The medicament will usually be supplied as part of a pharmaceutical composition. The pharmaceutical composition will desirably be provided in sterile form. It may be provided in unit dosage form and may also be provided in a sealed container. A plurality of unit dosage forms may be provided.

Pharmaceutical compositions may include one or more of the following: pharmaceutically carriers, preserving agents, solubilising agents, stabilising agents, wetting agents, emulsifiers, sweeteners, colorants, odorants, salts, buffers, coating agents, antioxidants, adjuvants, excipients and diluents. They may also contain other therapeutically active agents in addition to binding agents. Where two or more therapeutic agents are used they may be administered separately (e.g. at different times and/or via different routes) and therefore do not always need to be present in a single composition. Thus combination therapy is within the scope of the methods and compositions described here.

Pharmaceutical compositions may be provided in controlled release form. This can be achieved by providing a pharmaceutically active agent in association with a substance that degrades under physiological conditions in a predetermined manner. Degradation may be enzymatic or may be pH-dependent.

Pharmaceutical compositions may be designed to pass across the blood brain barrier (BBB). For example, a carrier such as a fatty acid, inositol or cholesterol may be selected that is able to penetrate the BBB. The carrier may be a substance that enters the brain through a specific transport system in brain endothelial cells, such as insulin-like growth factor I or II. The carrier may be coupled to the active agent or may contain/be in admixture with the active agent. Liposomes can be used to cross the BBB. WO91/04014 describes a liposome delivery system in which an active agent can be encapsulated/embedded and in which molecules that are normally transported across the BBB (e.g. insulin or insulin-like growth factor I or II) are present on the liposome outer surface. Liposome delivery systems are also discussed in U.S. Pat. No. 4,704,355.

A pharmaceutical composition may be adapted for administration by any appropriate route. For example, it may be administered by the oral (including buccal or sublingual), rectal, nasal, topical (including buccal, sublingual or transdermal), vaginal or parenteral (including subcutaneous, intramuscular, intravenous or intradermal) routes. Such a composition may be prepared by any method known in the art of pharmacy—by admixing one or more active ingredients with a suitable carrier, for example.

Different drug delivery systems can be used to administer pharmaceutical compositions, depending upon the desired route of administration. Drug delivery systems are described, for example, by Langer (Science 249:1527-1533 (1991)) and by Illum and Davis (Current Opinions in Biotechnology 2: 254-259 (1991)).

Two preferred routes of administration of a binding agent will now be considered in greater detail:

(i) Parenteral Administration

Pharmaceutical compositions adapted for parenteral administration include aqueous and non-aqueous sterile injectable solutions or suspensions. These may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water for injections, immediately prior to use. Extemporaneous injection solutions and suspensions may be prepared from sterile powders, granules and tablets.

(ii) Intravenous Administration

Pharmaceutical forms suitable for injectable use include sterile buffered aqueous solutions or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

Dosages of binding agents can vary between wide limits, depending upon the nature of the treatment, the age and condition of the individual to be treated, etc. and a physician will ultimately determine appropriate dosages to be used. However, without being bound by any particular dosages, a daily dosage of from 1 μg to 1 mg/kg body weight may be suitable. The dosage may be repeated as often as appropriate. If side effects develop, the amount and/or frequency of the dosage can be reduced, in accordance with good clinical practice. The principal active ingredients are compounded for convenient and effective administration in effective amounts with a suitable pharmaceutically acceptable carrier in unit dosage form. In the case of compositions containing supplementary active ingredients, the dosages are determined by reference to the usual dose and manner of administration of the said ingredients. For example, see Houghton A N, Chapman P B, Bajorin D F. Antibodies in cancer therapy: clinical applications. In: Devita V T Jr, Hellman S, Rosenberg S A, eds. Biologic Therapy of Cancer. Philadelphia, Pa.: Lippincott; 1991;533-549. In general, the optimum dose may be determined empirically in a clinical trial by administering escalating doses of antibody until the maximum tolerated dose is determined. The MTD is defined as the highest dose preceding that at which 50% of patients experience dose-limiting toxicity.

A further aspect is the provision of an image or model of a binding agent as described here. This may be computer-generated or may be physical. The image or model is preferably two or three-dimensional. It may be an X-ray crystallographic image or model and may comprise a plurality of co-ordinates. It may show one or more: binding sites, hydrophilic or hydrophobic regions, bonds, etc. For ease of viewing the image or model is preferably adapted for manipulation so to allow different parts to be viewed (e.g. by rotation, by zooming in or out, etc.).

A data carrier that comprises data for such an image or model is also within the scope of the methods and compositions described here, as is a computer that comprises said data or data carrier. Preferably the computer is set up to display the image or model.

The image or model is useful in predicting the structure and/or function of potential new therapeutic agents. For example, one or more changes may be made to the image or model and the effect(s) of those changes may be predicted or analysed.

A binding agent, image or model, data carrier, or computer as aforesaid is useful in a drug development program. A drug or drug candidate obtained via a drug development program in which the binding agent, image or model, data carrier, or computer is used is also within the scope of the methods and compositions described here.

Drugs or drug candidates are tested for activity and/or activity. We also provide a method comprising providing a binding agent, drug or drug candidate, as aforesaid, and testing the activity and/or toxicity of the binding agent, drug, or drug candidate in vivo or in vitro.

In order to aid in screening, testing, analysis, purification, etc., it may be desired to immobilise a binding agent as described here. We therefore provide the binding agent in immobilised form. Immobilisation may be achieved by various means, including the use of Staphylococcus protein A or of epitopes.

An immobilised binding agent may be provided as part of an organised array (e.g. an array having a grid-like structure). This can aid in identification when screening and is also useful in high throughput screening. The array may comprise or consist of a plurality of binding agents, each as described. It may be provided upon a generally planar surface.

The present invention will now be described by way of non-limiting examples.

### EXAMPLES

### Example 1

### Differentially Activated Bispecific (DAB) Antibody

One example of a binding agent is a differentially activated bispecific (DAB) antibody. This is a chimeric bispecific antibody designed for therapeutic removal of abnormal cells from the body.

Two Fab modules target the abnormal cells. Effectors are recruited by a human Fc module and also by an Fab module directed at effector cells. The construct is differentially active in the sense that initial binding to an effector cell leads to much less implementation of Fc-recruited effector function than does binding to the targeted cell. In this way damage to effector cells, and undesired symptoms due to cytokine release from effector cells in blood, are minimised.

Principles of Engineering of DAB Antibody

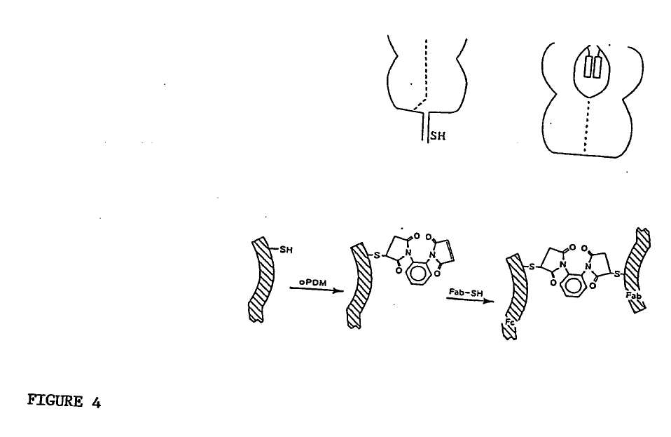

FIG. 4 illustrates fundamentals of engineering of a DAB antibody. Each module has its hinge-region interchain SS bonds reduced, and sometimes further manipulated by disulfide interchange, to leave one or more SH groups. One module (Fc in this example) is then exposed to a surplus of a bismaleimide linker, so that one end of the linker reacts with an SH group to form a thioether bond while the other end continues to display a reactive maleimide group. Unreacted linker is removed and the maleimide-displaying module is allowed to react with a second SH-displaying module (Fab in this case) to form a bimodular or higher order construct. The linking unit, of about 9 Å length, consists of thioether bonds on either side of an o-phenylenedisuccinimidyl group. If disulfide interchange has left some nascent SH groups, further modular additions are possible.

In certain embodiments of the DAB construct, an Fcγ module is deglycosylated enzymaticalt before being used as a building block.

### Example 2

### Structure and Properties of Un-Deglycosylated Differentially Activated Bispecific (DAB) Antibody

Structure

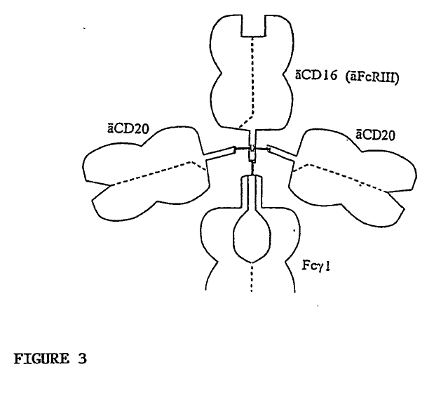

The structure of one form of a DAB (without deglycosylation) is given in FIGS. 3(a) and 3(b).

It is a tetramodular structure with all module-to-module connections involving cysteine residues at or near the hinge region. Thus the two anti-target Fab (anti-CD20) and the Fc modules are all connected to an anti-effector Fab (anti-CD16). The tandem thioether linkages are about 9 Å in length and are provided by bisuccinimidylphenyl groups linking to cysteinyl residues in the hinge of each module. There are 5 cysteines in each Fab module and 4 in the Fc module that can provide the sulphur for thioether bonds.

Properties

Some of the properties of the un-deglycosylated DAB are given below:

(a) Union with the target cell is stronger than union with the Fab-recruited effector cell.

(b) Union with the effector cell alone leaves access to docking sites on the Fc for the receptors FcRI, II, and III, and access to the docking site for complement, sterically hindered by the free anti-target Fab modules. This occurs by way of crowding of the two anti-target Fab and the Fc around the hinge of the anti-effector Fab. When union occurs with the target cell, the two anti-target Fab provide access to all docking sites on the Fc. This has been functionally verified in vitro by showing that DAB antibody+ effector cells only leads to minuscule damage to the effector cell in the presence of complement. In contrast DAB antibody+ target cells permits full complement damage to the target cells, while if effector cells are also present profound damage occurs to target cells due to recruitment by both Fc and anti-effector Fab.

(c) The combination of properties a) and b) above gives the DAB antibody substantial differential activity in inflicting much more Fc-mediated cell damage to target cells than to the effector cells that the DAB antibody recruits.

(d) Because the docking site for FcRn is some distance from the Fc hinge it is not expected to be sterically hindered while the anti-target Fab are free. Therefore the DAB antibody construct is likely to have a high half-life in human or animal subjects.

### Example 3

### Synthesis of Un-Deglycosylated Differentially Activated Bispecific Antibody

A brief outline of one method of synthesis is given below, using the specific example of a DAB antibody designed for therapy of B-cell lymphoma in man. The molecular target on the neoplastic B cells is the CD20 molecule and the molecule used for recruiting effector cells with Fab is CD16. The starting modules are:

(1) F(ab′γ)₂ from mouse monoclonal IgG2a 1F5 (anti-CD20)

(2) F(ab′γ)₂ from mouse monoclonal IgG1 3G8 (anti-CD16)

(3) Fcγ1 from human normal IgG

The method of synthesis is as follows:

(a) Fab′γ-maleimide (anti-CD20)

F(ab′γ)₂ ex 1F5 is reduced by 1 mM dithiothreitol (DTT) at pH 8.0 and the resulting Fab′γ (—SH)₅ separated by gel chromatography. It then reacts with ImM o-phenylenedimaleimide (PDM) at pH 5.0 to yield Fab′γ-maleimide, which is separated and concentrated by ion-exchange chromatography.

(b) Fab′γ (—SH)₅ (anti-CD16)

F(ab′γ)₂ ex 3G8 is reduced by 1 mM DTT at pH 8.0 and the resulting Fab′γ (—SH)₅ separated by gel chromatography.

(c) F(ab′γ)₃-maleimide

Fab′γ-maleimide_and Fab′γ (—SH)₅ are mixed in a ratio of 2.2:1 and allowed to react at pH 5.0. The resulting F(ab′γ)₃ is separated by gel chromatography and then reacted with PDM to yield F(ab′γ)₃-maleimide.

(d) Fcγ (—SH)₄

Fcγ1 is reduced by 1 mM DTT at pH 8.0 and the resulting Fcγ (—SH)₄ separated by gel chromatography.

(e) Fab₃Fc (DAB antibody)

F(ab′γ)₃ -maleimide and Fcγ (—SH)₄, are mixed at a mass ratio of 2:1 and allowed to react at pH 5.0. Finally the reaction mixture undergoes SS-interchange with 0.5 mM cysteine at pH 8.4 in order to close the Fc hinge, and the Fab₃Fc product is separated by gel chromatography.

### Example 4

### Properties of De-Glycosylated Differentially Activated Bispecific Antibody

(a) Union with an effector cell in the absence of a target cell leaves access to docking sites on the Fc for the receptors FcRI, II and III, and for complement, sterically hindered by free anti-target Fab modules, which are crowded with the Fc around the hinge of the anti-effector Fab module. Docking sites for the Fc receptors and complement are also impaired by the deglycosylation of the Fc. (Evidence to hand suggests that the site for FcRI, II and III is totally disabled but that a depleted site remains for complement.) The combination of these facts means that an effector cell coated with DAB antibody, in the absence of a target cell, is unlikely to be damaged by either (1) antibody-dependent cellular cytotoxicity due to other effector cells docking on the Fc module; or (2) complement-mediated cytotoxicity due to the component C1 docking on the Fc module.

(b) Union with an effector cell in the absence of a target cell is unlikely to crosslink the effector cell surface, because (1) the Fab modules are only univalent for the effector cell; (2) union with receptors FcRI, II and III on the surface of the coated effector cell is impaired for the same reasons as given above in relation to other effector cells; (3) the pH of extracellular fluid does not favour union with any FcRn receptor which may be present on the coated effector cell (see under d. below). The absence of crosslinking means that an effector cell coated with DAB antibody, in the absence of a target cell, is unlikely to release toxicity-inducing cytokines. (The danger of massive cytokine release is emphasized in: DM Segal et al. Bispecific antibodies in cancer therapy. Curr. Opin. Immunol. 11:558-562, 1999).

(c) Union with a target cell is bivalent and therefore stronger and more prolonged than isolated union with an effector. It will be followed by:

(1) A degree of complement-mediated cytotoxicity initiated by the component C1 docking on the Fc module. It has been found that deglycosylation reduces this phenomenon but does not eliminate it. Engagement of the two anti-target Fab modules appears to remove steric hindrance to C1 docking.

(2) Engagement by the anti-effector module of effector cells displaying the receptor FcRIII, chiefly NK cells and macrophages. Multiple anchoring will hold the effector cell tightly and the effective crosslinking of the FcRIII molecule will activate the cell for cytotoxicity. Crosslinking will also lead to cytokine release, enhancing the cytotoxicity and leading to inflammation in the vicinity of the targeted cells. If these events are widespread throughout the body some symptoms of toxicity will be inevitable, but the physician should be forewarned by the known mass and distribution of the targeted cells.

Cell death induced by effector cells recruited to the target cell surface is expected to be by far the most potent of the anti-tumour effects exerted by DAB antibody aimed at tumour. It is possible that the FcRIII-mediated cytotoxicity will be enhanced by the disablement of the Fc site for FcRI, II and III, because among the subtypes of FcRII is the inhibitory receptor FcRIIb whose engagement can considerably downgrade the activation of macrophages. Isolated disablement of FcRII in mice enhances the ability of antibody to destroy tumour (R A Clynes et al. Inhibitory Fc receptors modulate in vivo cytotoxicity against tumor targets. Nature Medicine 6:443-446, 2000).

(3) A degree of apoptosis (programmed cell death) induced by DAB antibody crosslinking the target cell surface and initiating signals leading to “death pathways”. This signaling is enhanced by the tethering of effector cells effectively increasing the degree of crosslinking of surface-bound antibody. (Shan et al. Apoptosis of malignant human B cells by ligation of CD20 with monoclonal antibodies. Blood 91:1644-1652, 1998.)

(d) The docking site for FcRn on the Fc module, being some distance from the Fc hinge, is not subject to steric hindrance from other modules, nor is it expected to be affected by deglycosylation of the Fc (Dorai et al. Aglycosylated chimeric mouse/human IgG1 antibody retains some effector functions. Hybridoma 10:211-217, 1991). DAB antibody is therefore expected to show prolonged metabolic survival, comparable to that of human IgG.

FcRn is present on many body cells, including the vascular endothelial cells thought to be a major site for catabolism of IgG. At the pH of extracellular fluid (7.4) Fcγ has no significant affinity for FcRn. On being endocytosed—a preliminary to catabolism—IgG is in endosomes of decreasing pH, histidines in the docking site for FcRn become protonated, and the IgG combines with FcRn on the endosomal wall. This diverts the molecule away from the path to destructive endolysosomes: instead it is trafficked back onto the cell surface where the higher pH leads to its release back into extracellular fluid.

### Example 5

### Synthesis of Deglycosylated Differentially Activated Bispecific Antibody

A deglycosylated Differentially Activated Bispecific Antibody (termed Fab₃Fcd) is made in a similar manner described above in Example 3, except that steps (d) and (e) are modified. The starting materials are identical, but the synthesis involves 2 major stages, the synthesis of Fab₃ and the attachment to it of deglycosylated Fc.

(a) Fab-maleimide (anti-CD20)

F(ab′γ)₂ ex 1F5 is reduced by 1 mM dithiothreitol (DTT) at pH 8.0 and the resulting Fab(—SH)₅ separated by gel chromatography. It then reacts with 1 mM o-phenylenedimaleimide (PDM) at pH 5.0 to yield Fab-maleimide, which is separated and concentrated by ion-exchange chromatography.

(b) Fab(—SH)₅ (anti-CD16)

F(ab□γ)₂ ex 3G8 is reduced by 1 mM DTT at pH 8.0 and the resulting Fab(—SH)₅ separated by gel chromatography.

(c) Fab₃-maleimide

Fab-maleimide and Fab(—SH)₅ are mixed in a ratio of 2.2:1 and allowed to react at pH 5.0. The resulting Fab₃ is separated by gel chromatography, then reacts with PDM to yield Fab₃-maleimide. The reaction is stochastic and Fab4 and Fab2 form as byproducts. During gel chromatography the latter is bled off, to be subjected to further reaction with Fab-maleimide.

(d) Fcd(—SH)₄

The Fc is first deglycosylated by reaction with the glycoamidase PNGaseF (AL Tarentino & THPlummer. Enzymatic deglycosylation of asparagine-linked glycans: purification, properties, and specificity of oligosaccharide-cleaving enzymes from Flavobacterium meningosepticum. Meth Enzymol 230:44-57, 1994). Any incompletely deglycosylated can be expected to display quite a variety of carbohydrate, so the completely deglycosylated (and therefore quite hydrophobic) Fc is separated and concentrated by hydrophobic interaction chromatography on Toyopearl TSK-butyl-650. The eluted deglycosylated Fcγ1 is designated Fcd.

Fcd is reduced by 1 mM DTT at pH 8.0 and the resulting Fcd(—SH)₄ separated by gel chromatography.

(e) Fab₃Fcd (DAB Antibody)

Fab₃-maleimide and Fcd(—SH)₄ are mixed at a mass ratio of 2:1 and allowed to react at pH 5.0. Finally the reaction mixture is alkylated with 10 mM iodoacetate at pH 8.0 in order to block surplus SH groups, and the Fab₃Fcd product is separated by gel chromatography.

Further Aspects

Paragraph 1. A binding agent comprising: (a) a first part that has one or more of the biological activities of an antibody Fc region when the binding agent is bound to a biological target (b) a second part that is capable of binding to the biological target with a valency of two or more; and (c) a third part that is capable of monovalent binding to an effector cell so that the effector cell can act upon the target when the second part is bound to the target.

Paragraph 2. A binding agent according to Paragraph 1, wherein the effector cell is capable of destroying, damaging, altering or removing the target.

Paragraph 3. A binding agent according to Paragraph 1 or Paragraph 2, wherein the target is deleterious to a human or non-human animal.

Paragraph 4. A binding agent according to Paragraph 3, wherein the target is a cancer cell or a part thereof.

Paragraph 5. A binding agent according to any preceding Paragraph, wherein the first part has one or more of the following biological activities when the binding agent is bound to a biological target: (a) complement activation; (b) induction or stimulation of phagocytosis by phagocytic cells; (c) antibody-dependent cellular cytotoxicity (ADCC); and (d) binding to the neonatal or Brambell Fc-receptor (FcRn).

Paragraph 6. A binding agent according to any preceding Paragraph, wherein at least one of the biological activities of the first part is modulated when the agent is bound to the target cell in comparison with when the agent is bound to the effector cell only.

Paragraph 7. A binding agent according to any preceding Paragraph, wherein at least one of the biological activities of the first part is at least ten times higher when the agent is bound to the target cell in comparison with when the agent is bound to the effector cell only.

Paragraph 8. A binding agent according to any preceding Paragraph, wherein, in the absence of binding of the second part to the target, the binding agent is configured so that at least one biological activity of the first part is prevented or reduced due to steric hindrance, and wherein said steric hindrance is removed or reduced when the second part binds to the target.

Paragraph 9. A binding agent according to Paragraph 8, wherein said at least one biological activity includes complement activation.

Paragraph 10. A binding agent according to Paragraph 8 or 9, wherein said at least one biological activity includes binding with FcRI, FcRII and/or FcRIII receptors.

Paragraph 11 A binding agent according to Paragraph any of Paragraphs 8 to 10, wherein endosomal binding to the first part so as to reduce lysosomal degradation of the binding agent in vivo is not prevented.

Paragraph 12. A binding agent according to Paragraph 11, wherein the first part comprises an FcRn docking site that is not sterically hindered in the absence of binding of the second part to the target.

Paragraph 13. A binding agent according to any preceding Paragraph, wherein the second part is capable of binding to a plurality of different targets or to a plurality of different parts of the same target.

Paragraph 14. A binding agent according to any preceding Paragraph comprising one or more Fab, Fab′ or F(ab′)₂ regions or parts thereof.

Paragraph 15. A binding agent according to any preceding Paragraph comprising one or more Fc regions, or parts thereof.

Paragraph 16. A binding agent according to any preceding Paragraph; comprising at least two anti-target Fab, Fab′ or F(ab′)₂ regions or parts thereof, at least one anti-effector cell Fab, or Fab′ regions or parts thereof, and at least one Fc region or a part thereof.

Paragraph 17. A binding agent according to any preceding Paragraph wherein the parts of the binding agent are derived from an IgG molecule.

Paragraph 18. A binding agent according to any preceding Paragraph wherein the parts are covalently linked together.

Paragraph 19. A binding agent according to any preceding Paragraph comprising one or more tandem thioether links that interconnect cysteine residues

Paragraph 20. A binding agent according to any preceding Paragraph wherein the second part binds specifically to the target.

Paragraph 21. A binding agent according to any preceding Paragraph wherein the second part has anti-CD 20 and/or anti CD-37 binding activity.

Paragraph 22. A binding agent according to any preceding Paragraph wherein the third part binds specifically to the effector cell.

Paragraph 23. A binding agent according to any preceding Paragraph, wherein the third part has anti-CD16 binding activity.

Paragraph 24. A binding agent according to any preceding Paragraph having a tetramodular structure, wherein two modules are capable of binding to a biological target, one module is capable of binding to an effector cell and another module has one or more of the biological activities of an antibody Fc region when the binding agent is bound to a biological target.

Paragraph 25. A binding agent according to any preceding Paragraph when bound to the effector cell.

Paragraph 26. A method of providing a binding agent according to any preceding Paragraph, comprising providing a plurality of modules and connecting them via tandem thioether linkages between cysteine residues.

Paragraph 27 A method according to Paragraph 26 wherein modules are linked via a maleimide linker (e.g. o-phenylenedimaleimide (PDM).

Paragraph 28. A binding agent according to any of Paragraphs 1 to 25, for use in medicine.

Paragraph 29. The use of a binding agent according to any of Paragraphs 1 to 25 in the preparation of a medicament for treating a disease or disorder caused by or involving the biological target.

Paragraph 30. The use according to Paragraph 29, wherein the disease or disorder is cancer.

Paragraph 31. The use according to Paragraph 30, wherein the disease or disorder is a lymphoma (e.g. a B-cell lymphoma).

Paragraph 32. The use according to Paragraph 29, wherein the disease or disorder is a pathogenic disease or disorder.

Paragraph 33. The use according to Paragraph 29, wherein the disease or disorder is an autoimmune disease or disorder.

Paragraph 34. A pharmaceutical composition comprising a binding agent according to any of Paragraphs 1 to 25; said composition optionally comprising a pharmaceutically acceptable carrier, diluent or excipient.

Paragraph 35. An image or model of a binding agent according to any of Paragraphs 1 to 25.

Paragraph 36. An image or model according to any of Paragraphs 1 to 25 that is computer generated.

Paragraph 37. A data carrier that comprises data for an image or model according to Paragraph 35.

Paragraph 38. A computer that comprises data for an image or model according to Paragraph 35 or 36, and/or that comprises a data carrier according to Paragraph 37.

Paragraph 39. A method comprising providing an image or model according to Paragraph 35 or 36, a data carrier according to Paragraph 37, or a computer according to Paragraph 38 and using it to predict the structure and/or function of potential new therapeutic agents.

Paragraph 40. A method comprising providing an image or model according to Paragraph 35 or 36 making one or more changes to it and, optionally, predicting or analysing an effect of those changes.

Paragraph 41. A drug development program that uses a binding agent according to Paragraph 1 to 25, an image or model according to Paragraph 35 or 36, a data carrier according to Paragraph 37, a computer according to Paragraph 38, or a method according to Paragraph 39 or 40.

Paragraph 42. A drug or drug candidate obtained or identified using a drug development program according to Paragraph 41.

Paragraph 43. A method comprising providing a binding agent according to any of Paragraphs 1 to 25, or a drug or drug candidate according to Paragraph 42 and testing in vivo or in vitro the activity and/or binding of the binding agent, drug, or drug candidate against a biological target.

Paragraph 44. A method comprising providing a binding agent according to any of Paragraphs 1 to 25, or a drug or drug candidate according to Paragraph 42 and testing in vivo or in vitro the toxicity of the binding agent, drug, or drug candidate.

Paragraph 45. A binding agent according to any of Paragraphs 1 to 25, or a drug or drug candidate according to Paragraph 42, when in immobilised form.

Paragraph 46. An array comprising a binding agent according to any of Paragraphs 1 to 25, or a drug or drug candidate according to Paragraph 42.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A binding agent comprising:
(a) a first part that comprises one or more of the biological activities of an antibody Fc region when the binding agent is bound to a biological target;
(b) a second part that is capable of binding to the biological target with a valency of two or more; and
(c) a third part that is capable of monovalent binding to an effector cell so that the effector cell can act upon the biological target when the second part is bound to the biological target.

2. A binding agent according to claim 1, in which the effector cell is capable of destroying, damaging, altering or removing the biological target.

3. A binding agent according to claim 1, in which the biological target is deleterious to a human or non-human animal.

4. A binding agent according to claim 1, in which the biological target is a cancer cell or a part thereof.

5. A binding agent according to claim 1, in which at least one of the biological activities of the first part is modulated when the binding agent is bound to the biological target in comparison with when the binding agent is bound to the effector cell only.

6. A binding agent according to claim 1, in which at least one of the biological activities of the first part is at least ten times higher when the binding agent is bound to the biological target in comparison with when the binding agent is bound to the effector cell only.

7. A binding agent according to claim 1, in which, in the absence of binding of the second part to the biological target, the binding agent is configured so that at least one biological activity of the first part is prevented or reduced due to steric hindrance, and in which the steric hindrance is removed or reduced when the second part binds to the biological target.

8. A binding agent according to claim 1, in which the first part comprises an FcRn docking site that is not sterically hindered in the absence of binding of the second part to the biological target.

9. A binding agent according to claim 1, in which the first part comprises one or more of the following biological activities when the binding agent is bound to a biological target: (a) complement activation; and (b) binding to the neonatal or Brambell Fc-receptor (FcRn).

10. A binding agent according to claim 9, which is modified to reduce activation of an effector cell in the absence of binding of the binding agent to a biological target.

11. A binding agent according to claim 9, which is modified to reduce binding to an FcRI, FcRII and/or an FcRIII receptor in the absence of binding of the binding agent to a biological target.

12. A binding agent according to claim 9, in which the first part comprises an Fc region which lacks one or more glycans normally associated with a natural Fc molecule.

13. A binding agent according to claim 12, in which the first part comprises an Fc region which is enzymatically deglycosylated, preferably with glycoamidase PNGaseF.

14. A binding agent according to claim 12, in which the first part comprises a recombinant Fc region in which the asparagine residue corresponding to position 297 of the IgG heavy chain is replaced with a non-gylcosylatable amino acid residue.

15. A binding agent according to claim 9, in which the first part further comprises one or more of the following biological activities when the binding agent is bound to a biological target: (c) induction or stimulation of phagocytosis by phagocytic cells; and (d) antibody-dependent cellular cytotoxicity (ADCC).

16. A binding agent according to claim 9, in which the at least one biological activity includes binding with FcRI, FcRII and/or FcRIII receptors.

17. A binding agent according to claim 1, in which endosomal binding to the first part so as to reduce lysosomal degradation of the binding agent in vivo is not prevented.

18. A binding agent according to claim 1, in which the second part is capable of binding to a plurality of different biological targets or to a plurality of different parts of the same biological target.

19. A binding agent according to claim 1 comprising one or more Fab, Fab′ or F(ab′)₂ regions or parts thereof.

20. A binding agent according to claim 1 comprising one or more Fc regions, or parts thereof.

21. A binding agent according to claim 1, comprising at least one anti-target Fab, Fab′ or F(ab′)₂ regions or parts thereof, at least one anti-effector cell Fab, or Fab′ regions or parts thereof, and at least one Fc region or a part thereof.

22. A binding agent according to claim 21, which comprises at least two anti-target Fab, Fab′ or F(ab′)₂ regions or parts thereof.

23. A binding agent according to claim 1, in which any one or more of the first, second and third parts of the binding agent are derived from an IgG molecule.

24. A binding agent according to claim 1, in which any one or more of the first, second and third parts are covalently linked to each other.

25. A binding agent according to claim 1 comprising one or more tandem thioether links that interconnect cysteine residues.

26. A binding agent according to claim 1, in which the second part binds specifically to the biological target.

27. A binding agent according to claim 1, in which the second part comprises anti-CD 20 and/or anti CD-37 binding activity.

28. A binding agent according to claim 1, in which the third part binds specifically to the effector cell.

29. A binding agent according to claim 1, in which the third part comprises anti-CD 16 binding activity.

30. A binding agent according to claim 1, having a modular structure, in which one modules is capable of binding to a biological target, one module is capable of binding to an effector cell and another module comprises one or more of the biological activities of an antibody Fc region when the binding agent is bound to a biological target.

31. A binding agent according to claim 30, comprising two modules capable of binding to the same biological target.

32. A binding agent according to claim 1, when bound to an effector cell.

33. A part, component or module for use in the manufacture of a binding agent according to claim 1.

34. A method of providing a binding agent according to claim 1, comprising providing a plurality of modules and connecting them via tandem thioether linkages between cysteine residues.

35. A method of providing a binding agent, comprising the steps of: (a) providing a first part comprising one or more of the biological activities of an antibody Fc region when the binding agent is bound to a biological target; (b) providing a second part capable of binding to the biological target with a valency of two or more; (c) providing a third part capable of monovalent binding to an effector cell so that the effector cell can act upon the biological target when the second part is bound to the biological target; and covalently joining the first, second and third parts.

36. A method according to claim 34, in which the modules or parts of the binding agent are as set out in any preceding claim.

37. A method according to claim 34, in which the modules or parts are linked via a maleimide linker (e.g. o-phenylenedimaleimide (PDM)).

38. A binding agent according to claim 1, for use in medicine.

39. The use of a binding agent according to claim 1 in the preparation of a medicament for treating a disease or disorder caused by or involving the biological target.

40. The use according to claim 39, in which the disease or disorder is selected from the group consisting of: cancer, a lymphoma (e.g. a B-cell lymphoma), an infectious disease or disorder and an autoimmune disease or disorder.

41. A pharmaceutical composition comprising a binding agent according to claim 1; the composition optionally comprising a pharmaceutically acceptable carrier, diluent or excipient.

42. An image or model, preferably a computer generated image or model, of a binding agent according to claim 1.

43. A data carrier that comprises data for an image or model according to claim 42.

44. A computer that comprises data for an image or model of a binding agent that comprises a data carrier according to claim 43.

45. A method comprising providing an image or model according to claim 42 and using it to predict the structure and/or function of potential new therapeutic binding agents.

46. A method comprising providing a data carrier according to claim 43 and using it to predict the structure and/or function of potential new therapeutic binding agents.

47. A method comprising providing a computer according to claim 44 and using it to predict the structure and/or function of potential new therapeutic binding agents.

48. A method comprising providing an image or model according to claim 42, making one or more changes to it and, optionally, predicting or analysing an effect of those changes.

49. A drug development program that uses a binding agent according to claim 1.

50. A drug development program that uses an image or model according to claim 42.

51. A drug development program that uses a data carrier according to claim 43.

52. A drug development program that uses a computer according to claim 44.

53. A drug development program that uses a method according to claim 45.

54. A drug or drug candidate obtained or identified using a drug development program according to claim 49.

55. A method comprising providing a binding agent according to claim 1 and testing in vivo or in vitro the activity and/or binding of the binding agent, drug, or drug candidate against a biological target.

56. A method comprising providing a drug or drug candidate according to claim 54 and testing in vivo or in vitro the activity and/or binding of the binding agent, drug, or drug candidate against a biological target.

57. A method comprising providing a binding agent according to claim 1 and testing in vivo or in vitro the toxicity of the binding agent, drug, or drug candidate.

58. A method comprising providing a drug or rug candidate according to claim 54 and testing in vivo or in vitro the toxicity of the binding agent, drug, or drug candidate.

59. A binding agent according to claim 1, when in immobilised form.

60. A drug or drug candidate according to claim 54, when in immobilised form.

61. An array comprising a binding agent according to claim 1, or a drug or drug candidate according to claim 48.

62. An array comprising a drug or drug candidate according to claim 54.

63. A method comprising the steps of: (a) exposing a Fc-containing polypeptide to a matrix; (b) allowing the Fc-containing polypeptide to bind to the matrix by a hydrophobic interaction; (c) removing the Fc-containing polypeptide from the matrix by disrupting the hydrophobic interaction.

64. A method of separating an Fc-containing polypeptide from other components in a sample, the method comprising: (a) exposing the sample to a matrix; (b) allowing the Fc-containing polypeptide to bind to the matrix by a hydrophobic interaction; and optionally removing one or more components of the sample by washing the matrix; and (c) removing the Fc-containing polypeptide from the matrix by disrupting the hydrophobic interaction.

65. A method according to claim 63, in which the matrix comprises Toyopearl TSK-butyl-650.

66. A method according to claim 64, in which the matrix comprises Toyopearl TSK-butyl-650.
